(19) United States
(12) Patent Application Publication
(10) Pub. No.: US 2026/0346476 A1
(43) Pub. Date: Nov. 5, 2026

(54) Tool for Use as a Micromanipulator and Method for Producing the Tool

(71) Applicant: 3D Microprint GmbH, Chemnitz (DE)

(72) Inventors: Michael Blau, Chemnitz (DE); Joachim Göbner, Bürgel (DE); Thomas Starke, Chemnitz (DE)

(21) Appl. No.: 19/118,041

(22) PCT Filed: Dec. 7, 2023
(86) PCT No.: PCT/DE2023/100947

§ 371 Date: Apr. 3, 2025

(30) Foreign Application Priority Data
Dec. 8, 2022 (DE) 202022106858.5
Dec. 6, 2023 (DE) 102023134213.4

(51) Int. Cl. B25J 15/00; B22F 10/20; B25J 19/00; B33Y 10/00; B33Y 80/00

(52) U.S. Cl. CPC B25J 15/0028; B22F 10/20; B25J 19/007; B33Y 10/00; B22F 2304/10; B33Y 80/00

(57) ABSTRACT

The invention relates to a tool for use as a micromanipulator and to a method for producing the tool. The tool consists of a plurality of parts in the form of—a frame (1) which is oriented along a longitudinal axis (L) and which is arranged as a hollow body around an interior,—at least one manipulator element (2) arranged on a first end face (1.1) of the frame (1), and—at least one actuation element (3) which is arranged on a second end face (1.2) opposite the first end face (1.1) and which is mounted slidably and axially movably in the interior of the frame (1), said parts being connected interlockingly and movably to one another by means of necessary connecting and bearing elements, and the tool being entirely generated by additive manufacture, including all the necessary gaps between the parts movably connected to one another. According to the method, the tool with all its parts and with the gaps between the movable parts is manufactured entirely by additive manufacture in one processing operation.

### CROSS REFERENCE TO RELATED APPLICATIONS

This application is the U.S. national stage of International Application No. PCT/DE2023/100947, filed on Dec. 7, 2023. The international application claims the priority of DE 202022106858.5 filed on Dec. 8, 2022 and the priority of DE 102023134213.4 filed on Dec. 6, 2023; all applications are incorporated by reference herein in their entirety.

### BACKGROUND

The present invention relates to a tool for use as a micromanipulator and a method for producing the tool according to the preamble of the first and fifteenth claims.

In principle, micromanipulators or grippers that are used as micromanipulators are an assembly of several individual parts that are treated as several nested components in 3D printing from a manufacturing point of view. The mobility and functionality of such grippers are usually created by welding, gluing, soldering, pressing and other joining processes. The production and assembly requires several individual steps and is complex and error-prone. The connection points of the individual components are also susceptible to soiling and/or wear

For example, DE 101 36 581 A1 discloses a method for measuring and/or controlling the gripping force of a micromanipulator with which objects with microscopic dimensions, in particular in the range between one micrometer and one millimeter, can be gripped, wherein the micromanipulator has two clamping jaws which are displaceable relative to one another and between which an object can be clamped with the application of a clamping force, wherein one of the clamping jaws 2 is designed as part of a spring tongue 3 which is set into natural oscillation, wherein the change in the oscillation during contact of the object 1 by the spring tongue 3 is observed, wherein a variable characterizing the clamping force is calculated from the change and wherein the gripping force is controlled on the basis of the characterizing variable.

EP 2 120 818 A1 describes a method for producing a surgical micromanipulator tip comprising a proximal mounting base, at least a first elbow and a distal manipulator finger which is connected to the mounting base via the first elbow and terminates at the free end of the tip. In addition, the mounting base comprises at least one distal segment. The first elbow and the manipulator finger together form a tip portion, and the invention is characterized in that the tip portion is uniformly tapered by sharpening.

A functional module for a surgical element is known from the publication DE 10 2008 051 866 B3, which has a forceps element. A hinged claw is hinged to a rigid claw which is firmly connected to a sleeve carrying an external thread which can be screwed into a corresponding internal thread of the shaft. The blade actuating rod opens distally into a guide piston, which has a radial guide projection that is guided in an axial slot in the guide sleeve. The guide piston has a recess into which the blade shaft of a cutting blade can be inserted.

DE 10 2006 050 469 A1 describes a gripper for tubular workpieces with a drive unit and two gripper arms that can pivot in relation to each other. This is intended to ensure reliable gripping of strongly curved, in particular tubular workpieces, wherein the gripper arms are equipped with pressure pieces for fixing the workpiece. The pressure pieces are made of a heat-resistant, in particular ceramic material. This solution is not intended for endoscopic applications.

A tool head for endoscopic applications is known from publication DE 20 2004019 910 U1. In this, the tool holder is formed by a shaped sheet metal section.

According to the publication DE 20 2005 005 406 U1, a medical instrument with a distal end region has jaw parts which are movable relative to one another and can be pivoted about a pivot pin, wherein the head of the pivot pin is designed as a polygon. The pivot pin has a bushing and a pin that can be inserted into it. The pin and bush are screwed or pressed together. Furthermore, a clamping nut can be arranged between them.

A fabric tong is disclosed in US 2020/0107873, in which, as with the aforementioned solutions, a large number of individual parts have to be assembled.

With all common micromanipulators, several individual components must therefore be manufactured and assembled together, which involves a great deal of effort and is prone to errors, for example with regard to tolerances that must be adhered to. There is also a risk of corrosion if different materials are used.

### SUMMARY

The invention is based on the object of creating an alternative to conventionally manufactured micromanipulators which can be manufactured in a single work step, thus eliminating the need for assembly work and which have a simpler and functionally reliable design with similar opening angles and installation spaces.

The object is solved by the features of the 1^{st} and 15^{th} claims. Further useful designs of the invention are the subject matter of the dependent claims.

### DETAILED DESCRIPTION

This object is solved by a tool for use as a micromanipulator, having a frame which is oriented along a longitudinal axis and which is designed as a hollow body around an interior. Arranged on a first end face, the tool according to the invention has at least one manipulator element. Arranged on a second end face opposite the first end face, the tool according to the invention has at least one actuating element which is mounted slidably and axially movable in the interior of the frame. According to the invention, the frame, the at least one actuating element and the at least one manipulator element are positively and movably connected to one another and the tool is produced entirely by additive manufacturing.

The micromanipulator is intended in particular for medical applications and should preferably be used for minimally invasive surgery (MIS).

The frame can be cylindrical or rectangular in its cross-sectional area. The manipulator element is functionally connected to the actuating element and is moved by it.

The frame, the at least one manipulator element and the at least one actuating element, including the gaps required between them, are additively manufactured from one and the same material.

The tool can be made of metallic material or ceramic or a combination thereof and produced by micro laser sintering. Preferred metals are stainless steel, surgical steel, titanium or other metals commonly used in medical technology. It is also conceivable that the tool is made of plastic.

Preferably, the manipulator element has at least two gripper jaws. The at least two gripper jaws are arranged in such a way that they can assume a closed state and an open state, wherein in the closed state they rest against each other with a respective contact surface. Objects can be gripped between the contact surfaces.

In a preferred embodiment, a first gripper jaw is movably connected to the frame and the actuating element, while a second gripper jaw is rigidly connected to the frame. Such an embodiment represents a variant that opens on one side.

In the single-sided opening variant, it may also be provided that the second gripper jaw is integrated into the solid body of the frame, i.e. to be formed in one piece with the frame.

In another preferred embodiment, both gripper jaws are movably connected to the frame and the actuating element. Such an embodiment represents a variant that opens on both sides.

It may be provided that a gripper jaw has a first recess in a connecting area, into which a first projection and/or a first pin of the frame engages. The first recess is arranged centrally in the connecting area of the respective gripper jaw. It may also be provided that a respective gripper jaw, in particular a gripper jaw which is movably connected to the frame and/or the actuating element, has a second recess in which a second projection and/or a second pin of the frame engages, and that a respective gripper jaw has a third recess in which a projection and/or a pin of the actuating element engages (actuating pin). The second and third recesses are each arranged in the edge area of the connecting area. When the actuating element is moved in the axial direction, a force is exerted on the respective gripper jaw(s) in operative connection with the actuating element and the respective gripper jaw(s) are moved.

Advantageously, the gripper jaws, which are movably connected to the frame and the actuating element, are arranged to rotate about an axis.

It is conceivable that the first recess and the first pin can be dispensed with.

The two gripper jaws can be arranged in one plane or slightly offset from each other, wherein an offset arrangement at least ensures that the contact surfaces of the gripper jaws rest on each other when closed.

It may also be provided that the tool has at least one position locking device for axially fixing the at least one manipulator element. Such a position locking device can, for example, be designed in the form of a movable and/or elastically resilient and/or rigid latch or projection.

Advantageously, the position locking device ensures that the at least one manipulator element does not detach from the pin via which it is connected to the frame and/or the actuating element.

The manipulator element can also preferably be designed as a scissor mechanism or a spreader. In this case, the scissor mechanism or the spreader are also movably connected to the actuating element.

Preferably, the actuating element has at least one opening for actuation, e.g. for a cable pull, on its exposed end face facing away from the frame. The actuating element can be set into an axial movement via an attached cable pull actuation.

It may be provided that the tool according to the invention is designed as a robot end effector.

Alternatively, the tool according to the invention can be operated hydraulically or pneumatically

Preferably, the tool according to the invention is suitable for use in microinvasive examinations and operations. Other conceivable areas of application include laboratory technology and space research.

Preferably, the tool according to the invention is additively manufactured by micro laser sintering.

The frame, the manipulator element and the actuating element can be made of the same material.

According to the method, the tool according to the invention is manufactured for use as a micromanipulator in that the tool with all its parts and with the gaps between the parts that can move relative to one another is manufactured completely by additive manufacturing in a single machining operation.

This is preferably done by micro laser sintering.

Starting from a longitudinal side of the tool, it is built layer by layer, wherein each layer is made of a metallic or ceramic powder with a particle size of less than 20 μm and then melted by laser and then solidified, wherein the powder is not melted in the areas of the gaps between the parts to be produced, which should be movable in relation to each other.

During micro laser sintering, the frame, the manipulator element and the actuating element are built layer by layer, with a maximum gap of 25 μm between them.

Advantageously, a first layer of a powder is first applied to a building platform, the tool is produced/built by micro laser sintering and the finished tool produced by micro laser sintering is then separated from the building platform by wire erosion or another separation process.

For the first time, the tool in the form of a micromanipulator with all its parts and with the gaps between the parts that move in relation to each other is manufactured completely according to the method in one machining process using additive manufacturing.

Advantageously, the tool according to the invention is designed as a functional assembly due to the possibilities of additive manufacturing as a single component, which consists of several functional groups that can move in relation to each other. This eliminates the need for several processing steps that are conventionally required to manufacture an assembly: Production of several individual parts, guaranteeing and adhering to production tolerances of the individual parts including quality assurance, logistical merging of the individual parts for assembly, assembly work and quality assurance work after assembly.

Basically, the gripper is an assembly of several individual parts which, from a manufacturing point of view, is treated as several nested components in 3D printing. The gripper is manufactured from metal using micro laser sintering (MLS). In the finished gripper, the individual solids are connected to each other exclusively via form fit and without an assembly or joining process to form a movable mechanism. This fundamentally distinguishes the gripper design from conventionally manufactured metal gripper mechanisms with similar opening angles and installation spaces, whose mobility and functionality are created by welding, gluing, soldering, pressing and other joining processes. All individual bodies and, in particular, the rotary and sliding connections are modeled according to the process-specific design rules for the MLS, so that a particularly small amount of play is created in the moving elements. This and the simultaneously low surface roughness of the components enable a low-play fit.

All internal functional surfaces of the pivot and shear joints and therefore their function can be used directly after printing and separating the assembly from the building platform without any further surface finishing.

The functional assembly in the form of the gripper is manufactured as a single component, which contains several parts that can move in relation to each other, thanks to the possibilities of 3D printing, preferably micro laser sintering. This eliminates several processing steps that are conventionally required to manufacture an assembly, such as
1. Production of several individual parts in compliance with the required tolerances, including quality assurance;
2. Logistical merging of the individual parts for assembly;
3. Assembly work;
4. Quality assurance work after assembly.

### BRIEF DESCRIPTION OF THE DRAWINGS

The invention is explained in more detail below with reference to exemplary embodiments and associated figures, without being limited to these, wherein:

FIG. 1 shows a top view of the tool;

FIG. 2 shows a side view of a tool according to the invention in the closed state;

FIG. 3 shows the longitudinal section A-A according to FIG. 1 in the closed state;

FIG. 4 shows the longitudinal section B-B according to FIG. 1 in the closed state;

FIG. 5 shows a side view of a tool according to the invention in the open state;

FIG. 6 shows the longitudinal section A-A according to FIG. 1 in the open state;

FIG. 7 shows the longitudinal section B-B according to FIG. 1 in the open state;

FIG. 8 shows a perspective view of a tool according to the invention in the partially opened state.

### DETAILED DESCRIPTION OF THE PREFERRED EMBODIMENTS

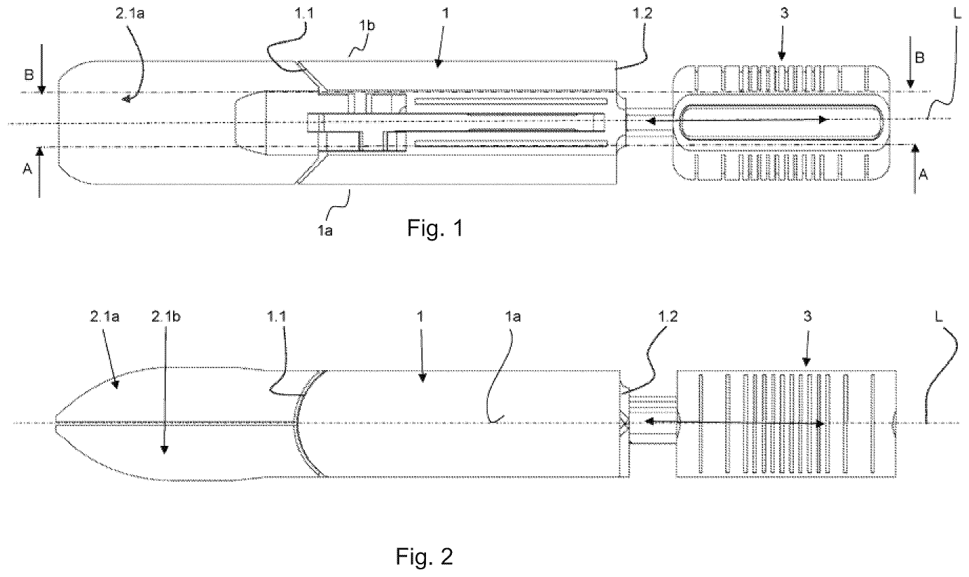

FIGS. 1 and 2 show a tool according to the invention in plan view and in side view in the closed state.

The tool has a frame 1, which is oriented along a longitudinal axis L. The frame 1 is designed as a hollow body with two opposing openings and an interior. The frame 1 has a first end face 1.1 and a second end face 1.2 opposite the first end face 1.1. The first end face 1.1 is adjoined by a manipulator element 2, which has two gripper jaws 2.1a and 2.1b in the embodiment shown. An actuating element 3 is arranged on the side of the second end face 1.2, which is axially displaceably mounted in the frame 1. The axial mobility of the actuating element 3 is indicated by a double-sided arrow. The actuating element 3 is operatively connected to the gripper jaws 2.1a and 2.1b.

FIGS. 1 and 2 show that the side surfaces 1a and 1b of the frame 1 are smooth and the bearing elements, which realize the pivoting of the gripper jaws 2.1a and 2.1b, are thus concealed and therefore located inside the tool.

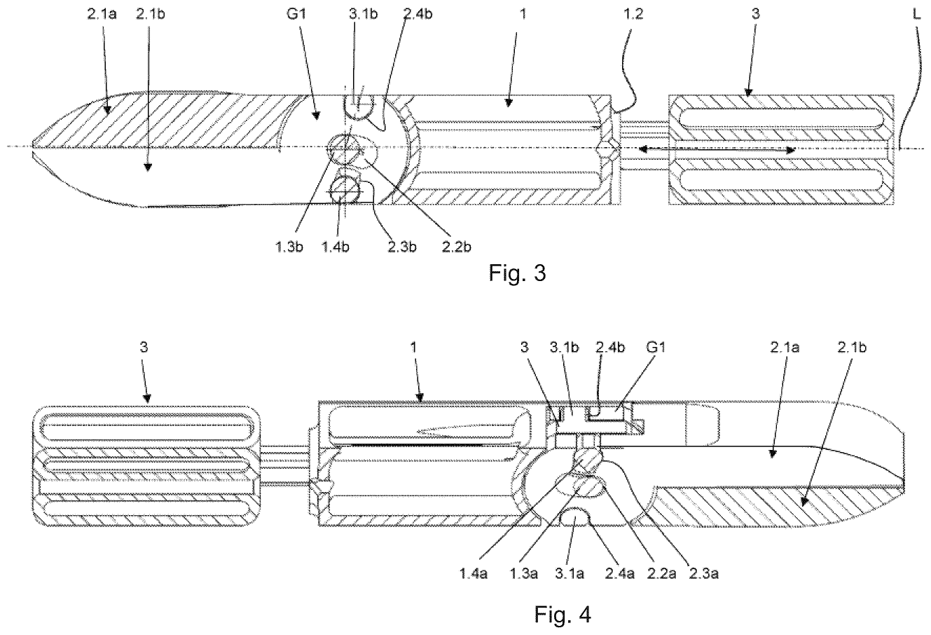

FIG. 3 shows the longitudinal section A-A according to FIG. 1 in the closed state from the direction of the longitudinal side 1a, although the lower gripper jaw 2.1b is not hatched. It can be seen that the lower gripper jaw 2.1b in this case is pivotably mounted by means of a joint G1, which is connected to the gripper jaw 2.1b in one piece. The joint G1 is also not hatched. The joint G1 is at least partially circular on its outer circumference.

The pivotably arranged lower first gripper jaw 2.1b has a curved first elongated hole 2.2b in the first joint G1, into which a first guide pin 1.3b arranged on the frame 1 transversely to the longitudinal axis L and pointing outwards engages. The movably arranged, here lower first gripper jaw 2.1b also has a first bearing recess 2.3b, into which a first bearing pin 1.4b arranged on the frame 1 engages. The movably arranged lower gripper jaw 2.1b also has a first recess 2.4b in its first joint area G1, into which an actuating pin 3.1b arranged on the actuating element 3 engages.

The first bearing recess 2.3b and the first mount 2.4b are arranged in an edge area of the joint area G1.

The lower first gripper jaw 2.1b is rotatably/pivotably mounted around the first bearing pin 1.4b.

The first guide pin 1.3b and the first bearing pin 1.4b are formed in one piece with the frame 1.

The first actuating pin 3.1b is formed in one piece with the actuating element 3.

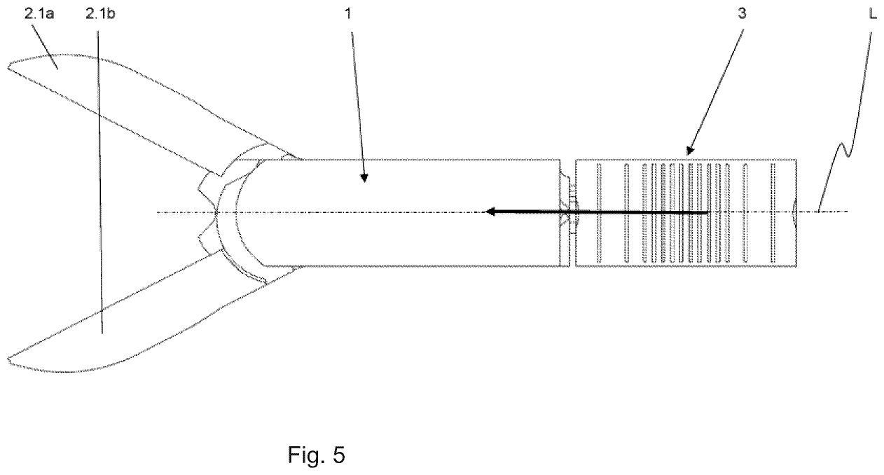

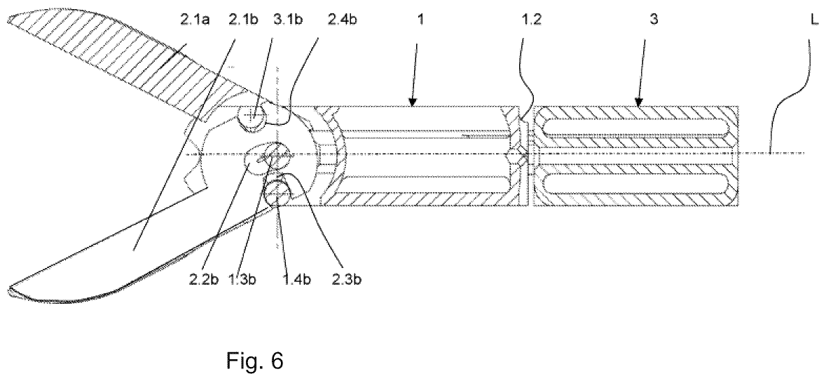

If the actuating element 3 is pushed in the direction of the frame 1, the first actuating pin 3.1b arranged at the front end of the actuating element 3, which engages in the first receptacle 2.4b of the joint G1, is also moved axially to the left here and thereby entrains the joint G1 and the associated gripper jaw 2.1b. The pivot point of the first joint G1 is located in the undesignated axis of the first bearing pin 1.4b. As a result, the lower gripper jaw 2.1b with the joint G1 pivots downwards around the first bearing pin 1.4b into the open position (see FIGS. 5 and 6).

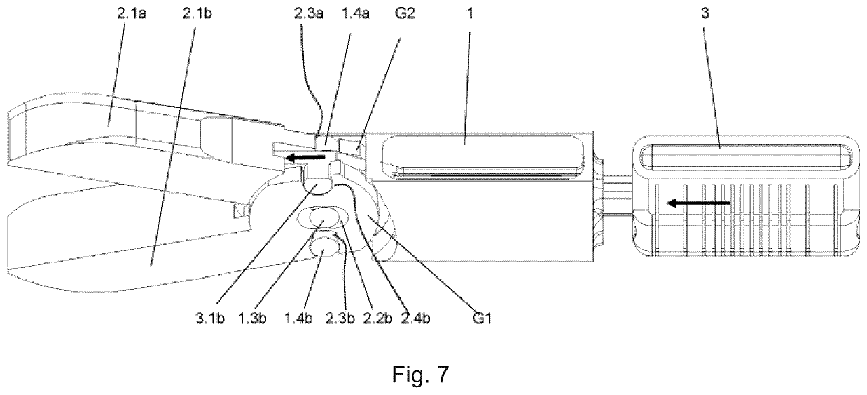

In order to pivot the upper second gripper jaw 2.1a upwards, a second joint G1 (not visible here) and
a second bearing pin 1.4a are located therein in a second bearing recess 2.3a of the second joint G2,
a second guide pin 1.3a is located in a second elongated hole 2.2b of the second joint G2 and
a second actuating pin 3.1a in a second receptacle 2.4a in the form of a recess in the second joint G2 in the region of a second joint G2 associated with the upper gripper jaw 2.1a (see FIGS. 4 and 7).

The second bearing pin 1.4a and the second guide pin 1.3a are formed in one piece with the frame 1.

The second actuating pin 3.1a is formed in one piece with the actuating element 3.

This design of the second joint G2 is rotated by 180° in relation to the longitudinal axis L. Therefore, the pivot point of the joint of the upper second gripper jaw 2.1a is at the top and the point of application of the second actuating pin 3.1a is at the bottom, as a result of which the upper second gripper jaw 2.1a is pivoted upwards when the actuating element 3 is moved in the direction of the frame 1.

FIG. 4 shows the closed position of the tool rotated transverse to the longitudinal axis L and from the other side in partial section. The second joint G2 and the second upper gripper jaw 2.1a are not hatched.

On the side of the tool shown here, the upper second gripper jaw 2.1a and the second joint G2 are formed in one piece and the frame 1 has a second actuating pin 3.1a at its end pointing in the direction of the gripper jaws 2.1a and 2.1b, which is aligned transversely to the longitudinal axis L and with which the joint G1 and the upper gripper jaw 2.1a can be actuated. For this purpose, the second actuating pin 3.1a engages in a recess or second receptacle 2.4a of the second joint G2.

The frame 1 has a second guide pin 1.3a, which is guided in a second elongated hole 2.2a, and a second bearing pin 1.4a is also provided on the frame 1, which is guided in a bearing recess 2.3a and about which the second joint G2 can pivot when the actuating element 3 is actuated.

If the actuating element 3 is moved in the direction of the frame 1 along the longitudinal axis L, the gripper jaws 2.1a and 2.1b are pivoted around the first and second bearing pins 1.4a and 1.4b by means of the first and second actuating pins 3.1a and 3.1b arranged on the actuating element 3 by a pivoting movement of the first and second joints G1, G2, thus opening the gripper jaws 2.1a and 2.1b.

FIG. 5 shows the side view of the tool in the open position and FIG. 6 shows the longitudinal section through plane A-A as shown in FIG. 1, also in the open position.

FIG. 5 shows, as in FIG. 2, that the actuating pins, bearing pins and guide pins as well as the receptacles, elongated holes and bearing recesses and joints cannot be seen from the outside, as they are concealed by the external material of the frame.

It can be seen from FIG. 6 that the actuating element 3 has been axially displaced in the direction of the frame 1 and the gripper jaws 2.1a and 2.1b are in an open position.

FIG. 6 shows that the first actuating pin 3.1b, which engages in the third first receptacle 2.4b, has pivoted the first joint area/first joint G1 about the first bearing pin 1.4b. The first joint G1 has moved with its first elongated hole 2.2 relative to the first guide pin 1.3b. The first guide pin 1.3b, which is mounted in the first elongated hole 2.2b, is used for stabilization.

FIG. 7 shows a three-dimensional representation of the tool in a partially open position. It can be seen from this that the second gripper jaw 2.1a at the top here has a second joint area/a second joint G2 in the direction of the frame 1, the pivot point of which is located about the undesignated axis of the second bearing pin 1.4a, which is arranged at the top here. The second bearing pin 1.4a is formed in one piece with the frame 1 and is mounted in a second bearing recess 2.3a of the joint G2.

The guide pins 1.3a, 1.3b and the bearing pins 1.4a, 1.4b are formed in one piece with the frame 1 and the actuating pins 3.1a, 3.1b are formed in one piece with the actuating element 3.

The two gripper jaws 2.1a and 2.1b have undesignated gripping surfaces that are to be aligned with each other and that rest against each other when closed. The gripping surfaces can be textured.

Preferably, the actuating element 3 has at least one undesignated opening for a push-pull element 3.2 on its exposed end face facing away from the frame 1. Tensile and compressive forces for opening and closing the gripper jaws 2.1a, 2.1b can be introduced via this. The actuating element 3 can thus be set into an axial movement via a corresponding actuation and the required push-pull movement can be realized via this.

Since the gripper jaws 2.1a, 2.1b are in a closed state, the contact surfaces resting on each other are not visible.

In order to move from a closed position of the gripper jaws to an open position, the actuating element is pushed in the direction of the frame 1. This causes both actuating pins 3.1a, 3.1b to move in the direction of the gripper jaws 2.1a, 2.1b and the joints G1, G2 are rotated about their bearing pins 1.4a, 1.4b.

As a result, the first gripper jaw 2.1b at the bottom of the figures pivots downwards with the first joint G1 around the first bearing pin 1.4b arranged at the bottom and the second gripper jaw 2.1a at the top pivots upwards with the second joint G2 around the second bearing pin 1.4a arranged at the top.

Preferably, such a gripper for medical applications is made of metal (e.g. steel, titanium). However, other materials suitable for laser sintering can optionally also be used, preferably for medical applications such as minimally invasive surgical applications.

All other features can be recognized adequately from FIGS. 1 and 2. Reference is hereby made to the corresponding passage in the description.

As already mentioned, all parts of the tool are manufactured using an additive manufacturing process, preferably by micro laser sintering in a single process sequence.

In micro laser sintering, the powder of the required material (metal, ceramic or plastic) is applied to a substrate plate using a device. A laser beam melts the areas of the powder that are to be solidified. The substrate plate is then lowered and another layer of powder is applied and melted by laser. This repetitive process produces the complete tool layer by layer.

After the manufacturing process and after separation from a substrate plate by wire erosion, the tool is ready for use with its movable parts, without any assembly work.

The tool in the form of the gripper intended for use as a micromanipulator has a diameter of 0.5 mm to 1.0 mm, preferably 0.7 mm to 0.9 mm, and an overall length of 1.2 mm to 1.7 mm, preferably 1.4 mm to 1.6 mm, for example, in the closed state in a small size.

In a larger design, the tool intended for use as a micromanipulator can, for example, have a diameter of 5.0 mm to 7.0 mm, preferably 5.5 mm to 6.5 mm, and an overall length of 12 mm to 20 mm, preferably 13 mm to 15 mm, when closed.

However, even larger dimensions are also possible, for example an overall length of 30 mm to 50 mm, preferably 35 mm to 45 mm and correspondingly larger diameters.

The dimensions of the tool in the form of the gripper/micromanipulator can of course be adapted to the necessary application conditions and requirements for minimally invasive surgery.

Such tools are also known as forceps in the field of medical technology.

### LIST OF REFERENCE NUMERALS

1 Frame
1.1 First end face of the frame
1a First side surface of the frame
1b Second side surface of the frame
1.2 Second end face of the frame
1.3a First guide pin
1.4b Second guide pin
1.5a First bearing pin
1.6b second bearing pin
2 Manipulator element
2.18 First gripper jaw/upper gripper jaw
2.1b Second gripper jaw/lower gripper jaw
2.2 First recess
2.2a Second elongated hole
2.2b First elongated hole
2.3a Second bearing recess
2.3b First bearing recess
2.4a Second receptacle/recess
2.4b First receptacle/recess
2.8 Connecting area
3 Actuating element
3.1a Second actuating pin
3.1b First actuating pin
3.2 Openings for push-pull element
G1 First joint
G2 Second joint
L Longitudinal axis

1. Tool for use as a micromanipulator, having a plurality of parts in the form
of a frame (1) which is oriented along a longitudinal axis (L) and is designed as a hollow body which is open at both ends,
at least one manipulator element (2) arranged on a first end face (1.1) of the frame (1), and
at least one actuating element (3), which is arranged on a second end face (1.2) opposite the first end face (1.1) and is mounted slidably and axially movable in the frame (1) along the longitudinal axis (L) and is operatively connected to the manipulator element,
wherein the parts in the form of the frame (1), the at least one actuating element (3) and the at least one manipulator element (2) are positively and movably connected to one another and the tool is produced entirely by additive manufacturing, including all necessary gaps between the movably connected parts.

2. Tool according to claim 1, characterized in that the frame (1), the at least one manipulator element (2) and the at least one actuating element (3) are made of one and the same material.

3. Tool according to claim 1, characterized in that the at least one manipulator element (2) has at least two gripper jaws (2.1a, 2.1b).

4. Tool according to claim 3, characterized in that a first gripper jaw (2.1a) is movably connected to the frame (1) and the at least one actuating element (3) and a second gripper jaw (2.1b) is rigidly connected to the frame (1).

5. Tool according to claim 3, characterized in that both gripper jaws (2.1a, 2.1b) are movably connected to the frame (1) and the at least one actuating element (3).

6. Tool according to claim 4, characterized in that the gripper jaw(s) (2.1a, 2.1b) movably connected to the frame (1) and the at least one actuating element (3) are arranged rotatably about an axis of a second bearing pin (1.4a, 1.4b) which is arranged transversely to the longitudinal axis (L) of the frame (1), wherein the second bearing pin (1.4a, 1.4b) is formed in one piece with the frame (1).

7. Tool according to claim 1, characterized in that the tool has at least one position locking device for axially fixing the at least one manipulator element (2).

8. Tool according to claim 1, characterized in that the at least one manipulator element (2) is designed as a scissor mechanism or as a spreader.

9. Tool according to claim 1, characterized in that the at least one actuating element has at least one opening (3.2) for actuation on its exposed end face.

10. Tool according to claim 1, characterized in that the tool is designed as a robot end effector.

11. Tool according to claim 1, characterized in that the frame (1), the manipulator element (2), the actuating element (3) and the actuating pins (3.1a, 3.1b), bearing pins (1.4a, 1.4b) and guide pins (1.3a, 1.3b) consist of the same material, wherein the bearing pins (1.4a, 1.4b) and guide pins (1.3a, 1.3b) are formed in one piece with the frame (1) and the actuating pins (3.1a, 3.1b) are formed in one piece with the actuating element (3).

12. Tool according to claim 1, characterized in that the tool is made of metal or a ceramic material.

13. Tool according to claim 1 for use in microinvasive examinations and operations.

14. Tool according to claim 1, characterized in that it is produced in a single process sequence by micro laser sintering.

15. Method for producing a tool for use as a micromanipulator according to claim 1, characterized in that the tool with the gaps between the moving parts is produced entirely by additive manufacturing in one machining process.

16. Method according to claim 14, characterized in that the tool is produced in a machining process by micro laser sintering.

17. Method according to claim 15, characterized in that, starting from a longitudinal side of the tool, the latter is built layer by layer, wherein each layer is applied from a metallic or ceramic powder with a particle size of less than 20 μm and then melted by laser and then solidified, wherein the powder is not melted in areas of the gaps between the parts to be produced which are intended to be movable with respect to each other.

18. Method according to claim 15, characterized in that during micro laser sintering the frame (1), the manipulator element (2) and the actuating element (3) are built layer by layer, wherein the gaps between them are at most 25 μm.

19. Method according to claim 15, characterized in that a first layer of a powder is applied to a building platform and the tool produced by micro laser sintering is then separated from the building platform by wire erosion or another separation process.
